(19) United States
(12) Patent Application Publication
(10) Pub. No.: US 2008/0161825 A1
(43) Pub. Date: Jul. 3, 2008

(54) Anatomical measurement tool

(75) Inventors: E. Skott Greenhalgh, Wyndmoor, PA (US); Stephen J. Kleshinski, San Jose, CA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 11/986,524

(22) Filed: Nov. 20, 2007

Related U.S. Application Data
(60) Provisional application No. 60/866,569, filed on Nov. 20, 2006.

(51) Int. Cl. A61B 17/58; A61M 31/00

(52) U.S. Cl. 606/102; 604/508; 604/509

(57) ABSTRACT

A measuring device for measuring tunnel defects in tissue is disclosed. The measuring device can size the defect to aid future deployment of a tissue distension device. Exemplary tunnel defects are atrial septal defects, patent foramen ovales, left atrial appendages, mitral valve prolapse, and aortic valve defects. Methods for using the same are disclosed.

### CROSS-REFERENCE TO RELATED APPLICATIONS

This application claims the benefit of U.S. Provisional Application No. 60/866,569, filed 20 Nov. 2006, which is incorporated by reference herein in its entirety.

### BACKGROUND OF THE INVENTION

1. Field of the Invention

The invention is related to the measuring devices and measurement of anatomical pathologies.

2. Description of the Related Art

The ability to accurately measure the dimensions of anatomical structures is of vital importance. In many cases, the anatomical geometry defines the treatment. A small object, small hole, or short length of anatomical pathology can go untreated because it has little to no clinical significance. Larger objects, holes, and longer length of anatomical pathology may lead to adverse clinical outcomes.

Additionally, many anatomical pathologies are treated with devices, including implantable devices, that are sized to fit the pathology. Knowledge of the specific size of the pathology aids the selection of an appropriately sized treatment device. Using trial and error techniques to determine the proper size of an implantable treatment device undesirably prolongs the surgical procedure, and fitting and removing improperly sized devices often further traumatizes the already-injured anatomical site.

Existing devices do not easily measure tunnel defects in soft tissue within body structures. Tunnel defects can be found in the heart (e.g., patent foramen ovale (PFO), left atrial appendage, mitral valve prolapse, aortic valve defects). Tunnel defects can be found through out the vascular system (e.g., venous valve deficiency, vascular disease, vulnerable plaque, aneurysms (e.g., neurovascular, abdominal aortic, thoracic aortic, peripheral). Tunnel defects can be found in non vascular systems (e.g., stomach with GERD, prostate, lungs).

A device for measuring the width of a distended defect in tissue is disclosed. The device has a longitudinal axis. The device can have a first elongated member. The first elongated member can be configured to expand away from the longitudinal axis. The device can have a second elongated member. The first elongated member can be opposite with respect to the longitudinal axis to the second elongated member. The second elongated member can be configured to expand away from the longitudinal axis. The device can have a lumen, for example, in a catheter. The device can have a porous cover on the lumen.

A method for sizing a tunnel defect. The method can include inserting a measurement tool into the tunnel defect. The method can include distending the tunnel defect into a distended configuration. The method can include measuring the tunnel defect in the distended configuration. Distending can include radially expanding the measurement tool. Measuring can include bending the first measuring wire around a front lip of the tunnel defect. Measuring can include emitting a contrast fluid in the tunnel defect.

### BRIEF SUMMARY OF THE INVENTION

Tissue distension devices can be deployed to tunnel defects in tissue. The tissue distension devices can be used to substantially close tunnel defects to treat, for example, patent foramen ovale (PFO), left atrial appendage, mitral valve prolapse, aortic valve defects. Examples of tissue distension devices include those disclosed in U.S. patent application Ser. No. 10/847,909, filed 19 May 2004; Ser. No. 11/184,069, filed 19 Jul. 2005; and Ser. No. 11/323,640, filed 3 Jan. 2006, all of which are incorporated by reference herein in their entireties.

To select a properly fitting tissue distension device, a measuring tool can first be deployed to measure the size of the tunnel defect. The tunnel defect can be measured in a relaxed or distended configuration. The tunnel defect can be distended by the measuring tool before or during measurement.

### BRIEF DESCRIPTION OF THE DRAWINGS

FIG. 1 illustrates a variation of the measurement tool in a first configuration.

FIGS. 2a, 2b, 3a and 3b illustrate variations of cross-section A-A of FIG. 1.

FIGS. 4 and 5 illustrate various embodiments of cross-section B-B of FIG. 1.

FIGS. 5 through 11 and 13 through 16 illustrate variations of the measurement tool in a second configuration.

FIG. 12 is a close-up view of the a portion of the measurement tool of FIG. 11 including the first measuring wire only, for illustrative purposes, transforming from a radially contracted to a radially expanded configuration.

FIGS. 17 through 27 illustrate variations of the measuring wire.

FIG. 28 illustrates a variation of cross-section C-C of FIG. 27.

FIG. 29 illustrates a variation of cross-section D-D of FIG. 27.

FIG. 30 illustrates a variation of a wire assembly.

FIG. 31 illustrates a variation of a wire sub-assembly.

FIG. 32 illustrates a variation of a wire assembly.

FIG. 33 illustrates a variation of a wire sub-assembly.

FIG. 34 illustrates a variation of a wire assembly.

FIGS. 35-37 illustrate variations of the measurement tool.

FIGS. 38a and 38b illustrate various sections of tissue having a tunnel defect.

FIG. 39 illustrates the tunnel defect of FIG. 38a or 38b.

FIGS. 40 through 42 illustrate a variation of a method for deploying an embodiment of the measurement tool.

FIGS. 43 and 44 illustrate a variation of a method for using various embodiments of the measurement tool.

FIG. 45 illustrates a variation of a method for using a variation of the measurement tool.

FIGS. 46 and 47 illustrate a variation of a method for using a variation of the measurement tool.

FIGS. 48 and 49 illustrate a variation of a method for using a variation of the measurement tool.

### DETAILED DESCRIPTION

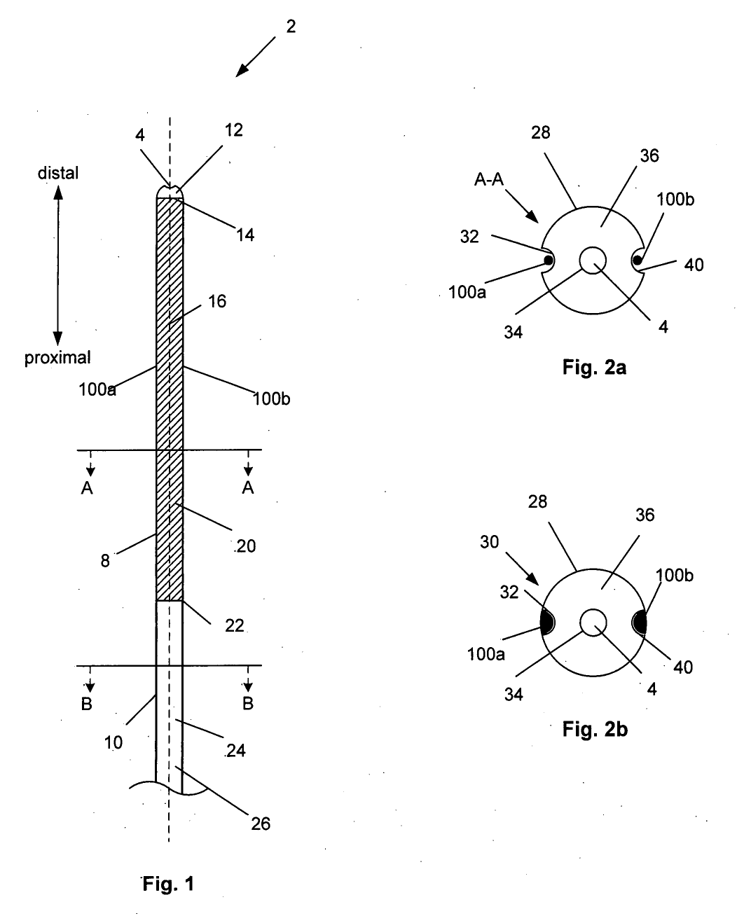

FIG. 1 illustrates an anatomical measurement tool 2, such as a tool for measuring the width in a relaxed and/or distended configuration of a tunnel defect 156 in tissue 154, in a radially contracted configuration. The measurement tool 2 can have a longitudinal axis 16. The anatomical measurement tool 2 can have a catheter 26, a first measuring wire 100a, and a second measuring wire 100b. The measuring wires 100 can be deformable, resilient, or combinations thereof over the length of the measuring wires 100.

The catheter 26 can have a catheter porous section 20. The catheter 26 can be entirely substantially non-porous. The catheter 26 can have a catheter non-porous section 24. The catheter porous section 20 can partially or completely circumferentially surround the catheter 26. The catheter porous section 20 can have holes or pores in the catheter outer wall 28. The pores can have pore diameters from about 1 μm (0.04 mil) to about 1 mm (0.04 in.), more narrowly from about 2 μm (0.08 mil) to about 300 μm (10 mil), for example about 150 μm (6.0 mil).

The first 100a and second measuring wires 100b can each have at least one wire radially constrained section 10 and at least one wire radially unconstrained section 8. The measuring wires 100 can transition from the wire constrained sections 10 to the wire radially unconstrained sections 8 at the wire proximal sheath ports 22. The first 100a and second measuring wires 100b between the wire proximal sheath ports 22 and the wire distal anchor 14 can be the radially unconstrained sections 8. The measuring wires 100 can be distally fixed to the catheter 26 at a wire distal anchor 14. The wire distal anchor 14 can be a hinged or otherwise rotatable attachment, for example, to allow the measuring wire 100 to rotate away from the longitudinal axis 16 at the wire distal anchor 14 during use.

The measurement tool 2 can have a tip 12 extending from a distal end of the catheter 26. The tip 12 can be blunt or otherwise atraumatic (e.g., made or coated with a softer material than the catheter 26, made with a soft substantially biocompatible rubber tip 12). A guide lumen 4 can extend from the tip 12. The guide lumen 4 can be configured to slidably receive a guidewire 170. The guide lumen 4 can exit through a dimple in the tip 12. The tip 12 need not be dimpled at the exit of the guide lumen 4.

FIG. 2a illustrates that the catheter 26 can have a catheter outer wall 28. The catheter outer wall 28 can be porous, or non-porous, or partially porous and partially non-porous. The catheter 26 can have a fluid lumen 36. The guide lumen 4 can be configured central to the cross-section of the catheter 26 or offset from the center of the cross-section, for example attached to the catheter outer wall 28. The guide lumen can have a guide lumen wall 34.

The first measuring wire 100a can removably and slidably reside in or removably and slidably attach to a recessed or raised first track 32 in the catheter outer wall 28. The second measuring wire 100b can removably and slidably reside in or removably and slidably attach to a recessed or raised second track 40 in the catheter outer wall 28.

To transform the measurement tool 2 from the radially contracted configuration to the radially expanded configuration, the first 100a and second measuring wires 100b in the wire radially constrained section 10 can be longitudinally translated, as shown by arrows 54 (not shown in FIG. 2a), in a distal direction. The first and second wires, for example, rotatably fixed at the wire distal anchor 14 and not radially constrained between the wire proximal sheath ports 22 and the wire distal anchor 14, can translate, as shown by arrows 52 (not shown in FIG. 2a), radially outward from the longitudinal axis 16.

FIG. 2b illustrates that the measuring wires 100 can have a configuration substantially equivalent to the configuration of the respective track 32 or 40. The measuring wires 100 and catheter 26 can be configured to create a substantially smooth, flush, regular configuration to the radial exterior cross-section (e.g., at A-A) of the measurement tool 2 when the wires 100 are in a contracted configuration. For example, the radially exterior cross-section (e.g., at A-A) of the measurement tool 2 can be configured substantially as a circle when the wires 100 are in a contracted configuration.

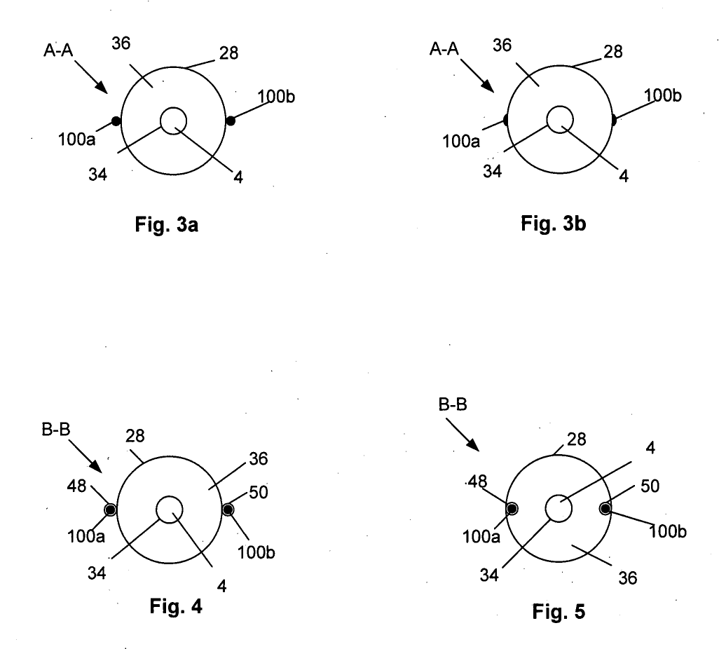

FIG. 3a illustrates that the first 100a and second measuring wires 100b in the wire radially unconstrained section 8 can be adjacent to, and reside on or attach to, the catheter outer wall 28. The catheter outer wall 28 can have no tracks for the measuring wires 100.

FIG. 3b illustrates that the measuring wires 100 can have a low-profile configuration. The low-profile configuration can have a cross-sectional configuration (e.g., at A-A) of a semi-circle, crescent, arc, oval, rectangle, or combinations thereof. The low-profile configuration can have a larger angular dimension than radial dimension, when measured with respect to the substantial center of the measurement device in the longitudinal direction. The measuring wires 100 and catheter 26 can be configured to create a substantially smooth, flush and regular exterior surface of the measurement tool 2 when the wires 100 are in a contracted configuration.

FIG. 4 illustrates that the first 100a and second measuring wires 100b can be slidably attached to and/or encased by first 48 and second sheaths 50, respectively. The interior of the sheaths can be coated with a low-friction material (e.g., polytetraflouroethylene (PTFE), such as Teflon® by E.I. du Pont de Nemours and Company, Wilmington, Del.).

FIG. 5 illustrates that the first sheath 48 and/or the second sheath 50 can be inside the catheter 26 (i.e., radially interior to the catheter outer wall 28).

The wire distal anchor 14 and wire sheaths 48 and/or 50 can be fixedly attached to the catheter 26. The wire distal anchor 14 and wire sheaths 48 and/or 50 can be slidably attached to the catheter 26.

The catheter outer wall 28 can be porous and/or non-porous, for example at different lengths along the catheter 26. For example, the catheter outer wall 28 in FIGS. 3a and 3b can be porous and the catheter outer wall 28 in FIGS. 4 and 5 can be non-porous.

The catheter 26 and/or tip 12 can have a stop. The stop can be longitudinally fixed with respect to the catheter 26 and/or the tip 12. The stop can be the tip 12, for example if the diameter of the tip 12 is larger than the diameter of the wire distal anchor 14. The stop can be configured to interference fit against the wire distal anchor 14 when the wire distal anchor 14 is distally translated beyond a maximum translation point with respect to the catheter 26 and/or tip 12.

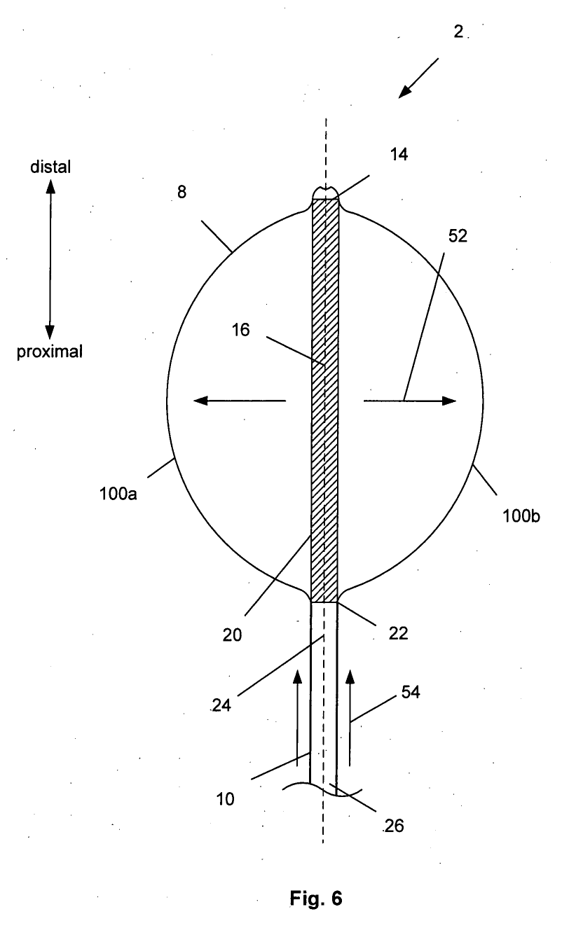

FIG. 6 illustrates the measurement tool 2 in a radially expanded configuration. The first 100a and second measuring wires 100b in the wire radially unconstrained section 8 can bow, flex, or otherwise be radially distanced or translate, as shown by arrows 52, with respect to the longitudinal axis 16 from the catheter 26. The first 100a and second measuring wires 100b can expand in a single plane (i.e., be coplanar).

The measuring wires 100 can be longitudinally translated, as shown by arrows 54, in the wire radially constrained sections 10. The first 100a and second measuring wires 100b in the wire radially unconstrained sections 8 can be radially expanded or otherwise translated, as shown by arrows, away from the catheter 26 (e.g., longitudinal axis 16) into a radially expanded configuration, for example by distally translating the measuring wires 100 in the wire radially constrained sections 10. The first 100a and second measuring wires 100b in the wire radially unconstrained sections 8 can be radially contracted or otherwise translated toward the catheter 26 (e.g., longitudinal axis 16) into a radially contracted configuration, for example by proximally translating the measuring wires 100 in the wire radially constrained section 10.

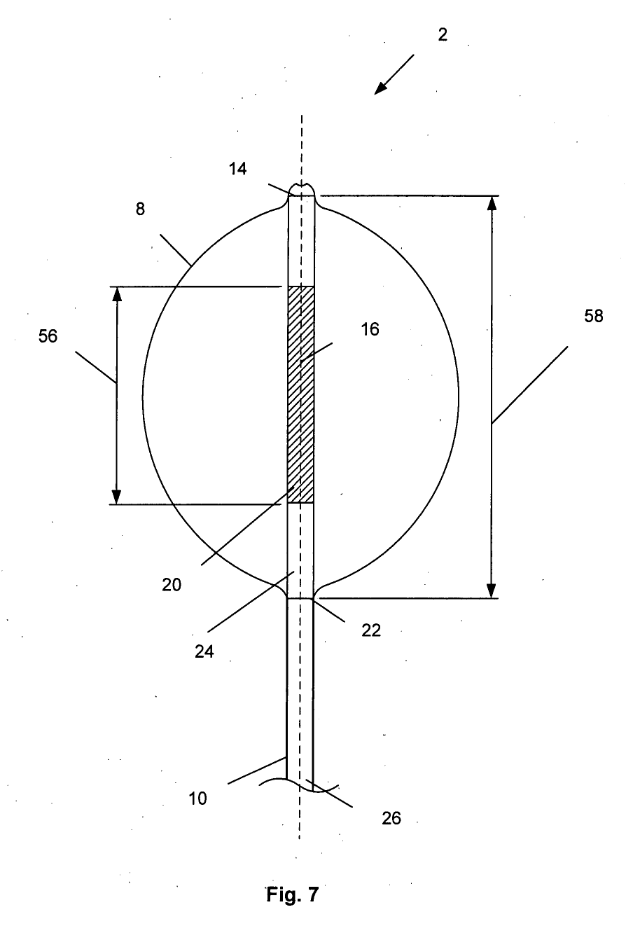

FIG. 7 illustrates that the catheter porous section 20 can have a porous section length 56. The longitudinal distance between the wire distal anchor 14 and the wire proximal sheath ports 22 (i.e., the wire radially unconstrained section 8) can be an unconstrained wire longitudinal length 58. The unconstrained wire longitudinal length 58 can be less than, substantially equal to (as shown in FIGS. 1 and 6), or greater than (as shown in FIG. 7) the catheter non-porous section 24.

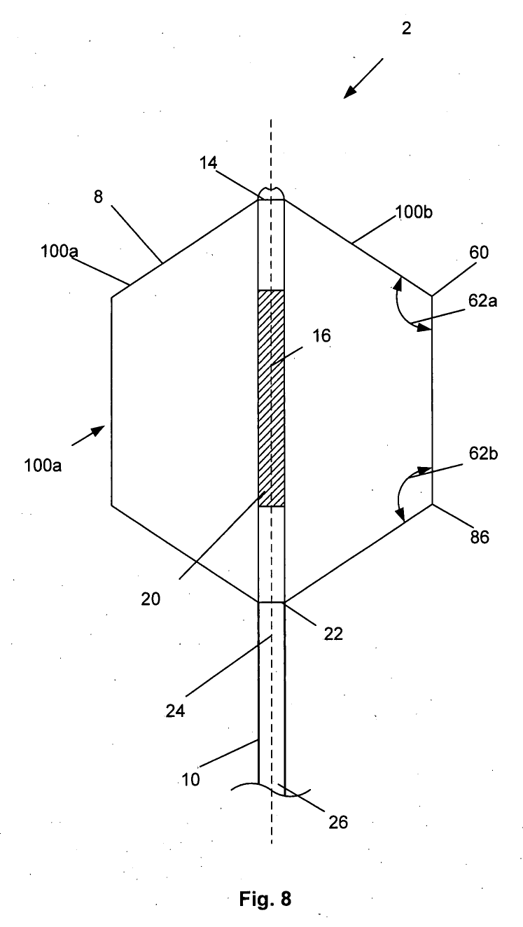

FIG. 8 illustrates that the first and second wires can have substantially discrete angles when the wires are in the radially expanded configurations. Each wire 100 can have a wire first hinge point 60 and a wire second hinge point 66. The wire hinge points 60 and/or 66 can be biased (e.g., before the measurement tool 2 is configured in the first configuration) to bend when the tension on the measuring wire 100 is decreased. The wire hinge points 60 and/or 66 can have hinges 106, bends, seams, links, other articulations, or combinations thereof.

The wire first hinge point 60 can have a wire first hinge angle 62a. The wire second hinge point 86 can have a wire second hinge angle 62b. In a radially expanded configuration, the wire hinge first and second angles 62a and 62b can be from about 10° to about 170°, more narrowly from about 30° to about 150°, yet more narrowly from about 45° to about 135°, for example about 125°. The wire hinge angle 62 when the measurement tool 2 is in a radially expanded configuration can be equivalent to the hinge angle 62, described infra, when the measurement tool 2 is in a radially contracted configuration.

FIG. 9 illustrates that the measurement tool 2 can have about 12 measuring wires 100. The measuring wires 100 can be radially expandable in a configuration where the first measuring wire 100a deploys substantially longitudinally adjacent to a third measuring wire 100c. The measuring wires 100 can be radially expandable in a configuration where the second measuring wire 100b deploys substantially longitudinally adjacent to a fourth measuring wire 100d.

The measuring wires 100 can each have a unique or paired longitudinal position for their wire proximal sheath ports 22 and wire distal anchors 14. For example, the first 100a and second measuring wires 100b can exit from wire first proximal sheath ports 22a (not shown on FIG. 9) and can be fixed at wire first distal anchors 14a (not shown on FIG. 9). The third 100c and fourth measuring wires 100d can exit from wire second proximal sheath ports 22b (not shown on FIG. 9) and can be fixed at wire second distal anchors 14b (not shown on FIG. 9). The wire first distal anchors 14a can be distal to the wire second distal anchors 14b. The wire first proximal sheath ports 22a can be at a substantially equivalent longitudinal position to the wire second distal anchors 14b. The wire second distal anchors 14b can be distal to the wire second proximal sheath ports 22b. This longitudinal spacing of the wire distal anchors 14 and wire proximal sheath ports 22 can be used for all of the measuring wires 100.

The measuring wires 100 on each side of the catheter 26 (e.g., the first, third, fifth, seventh, ninth and eleventh measuring wires or the second, fourth, sixth, eighth, tenth and twelfth measuring wires) can pass through the same or different sheaths.

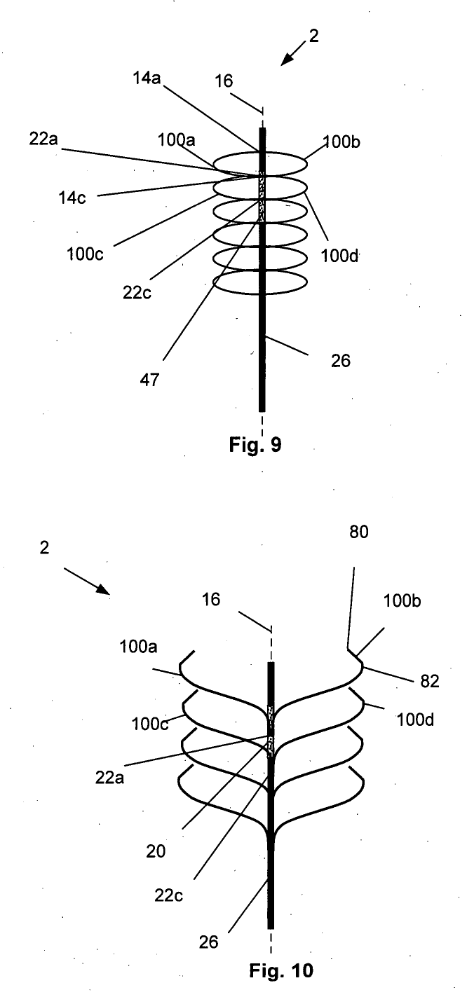

FIG. 10 illustrates that the measuring wires 100 can have distal ends that are not attached to the catheter 26 when the measuring wires 100 are in radially expanded configurations. Any or all measuring wire 100 can have a terminal end 80. When the measurement-tool 2 is in a radially expanded configuration, the terminal ends 80 can be unattached to the catheter 26. When the measurement tool 2 is in a radially expanded configuration, the measuring wires 100 can have a medial turn 82, bend, hinge 106, or otherwise angle medially between the terminal ends 80 and the wire proximal ports. A length of the measuring wires 100 can be biased to turn or bend medially when that length of the measuring wire 100 is in a relaxed configuration. The measurement tool 2 can have about eight measuring wires 100.

FIG. 11 illustrates that the measuring wires 100 can form a substantially circular or oval loop when the measuring wire 100 is in the radially expanded configuration. The measurement tool 2 can have six measuring wires 100. Each measuring wire 100 can have a separate proximal sheath port 22 (e.g., first, second, third, fourth, fifth and sixth proximal sheath ports 22a, 22b, 22c, 22d, 22e, and 22f), and wire distal anchors 14 (e.g., wire first, second, third, fourth, fifth and sixth distal anchors 14a, 14b, 14c, 14d, 14e and 14f)

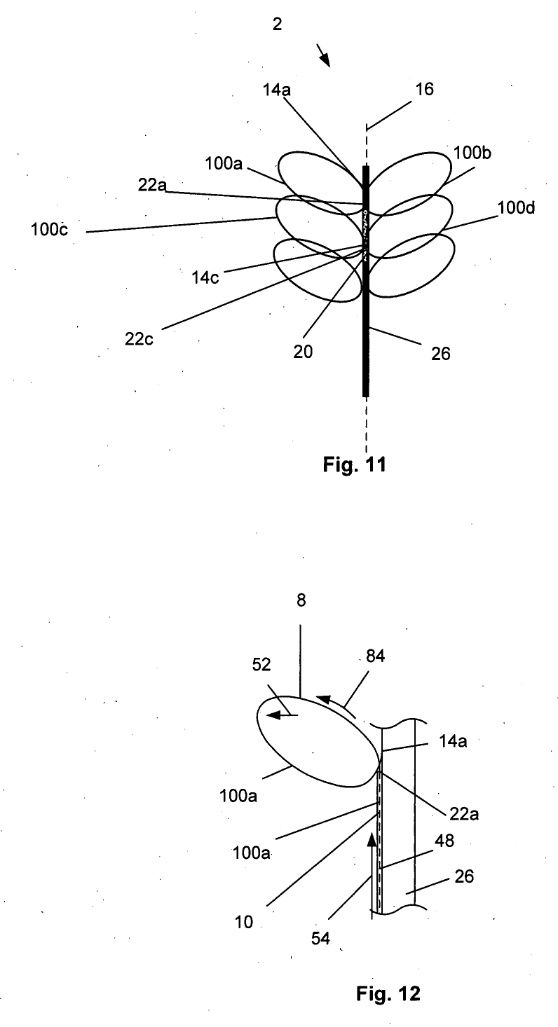

FIG. 12 illustrates that the loop of wire radially unconstrained section 8 can expand when the measuring wires 100 transform from the radially contracted configuration to the radially expanded configuration. The measuring wires 100 can be longitudinally translated, as shown by arrow 54, in the wire radially constrained sections 10. Along the length of the measuring wires 100 near the wire proximal port, the measuring wires 100 can translate along the longitudinal wire-axis, as shown by arrow 84. The measuring wires 100 in the wire radially unconstrained sections 8 can be radially expanded or otherwise translated, as shown by arrow 52, away from the catheter 26 (e.g., longitudinal axis 16) into a radially expanded configuration, for example by distally translating the measuring wires 100 in the wire radially constrained sections 10. The measuring wires 100 in the wire radially unconstrained sections 8 can be radially contracted or otherwise translated toward the catheter 26 (e.g., longitudinal axis 16) into a radially contracted configuration, for example by proximally translating the measuring wires 100 in the wire radially constrained section 10.

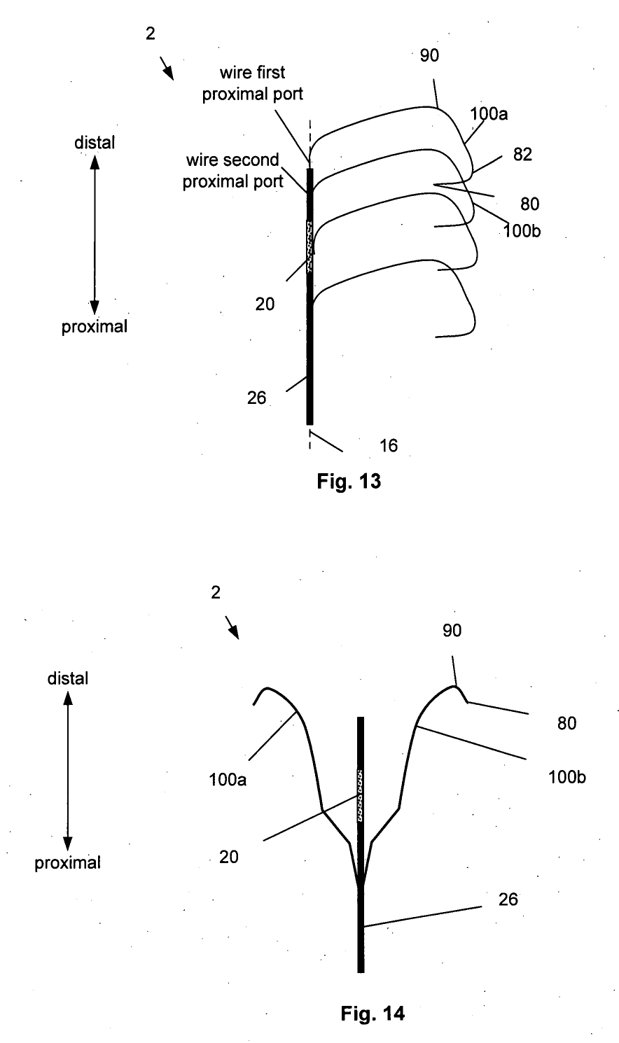

FIG. 13 illustrates that the measuring wires 100 can exit from the respective wire sheaths at the respective wire proximal ports. The measuring wires 100 can all exit the wire proximal ports on the same side of the catheter 26, or immediately turn to the same side of the catheter 26 after exiting the proximal wire ports. When the measurement tool 2 is in a radially expanded configuration, the measuring wires 100 can have a proximal turn, bend, hinge 106, or otherwise angle proximally after exiting the proximal wire port. When the measurement tool 2 is in a radially expanded configuration, the measuring wires 100 can have a medial turn 82, bend, hinge, or otherwise angle toward the longitudinal axis 16, for example, between the proximal bend 90 and the terminal end 80. Any length of the measuring wires 100 can be biased to turn or bend when that length of the measuring wire 100 is in a relaxed configuration. FIG. 14 illustrates that the measuring wire 100 can have a proximal turn, bend, hinge 106, or otherwise angle proximally.

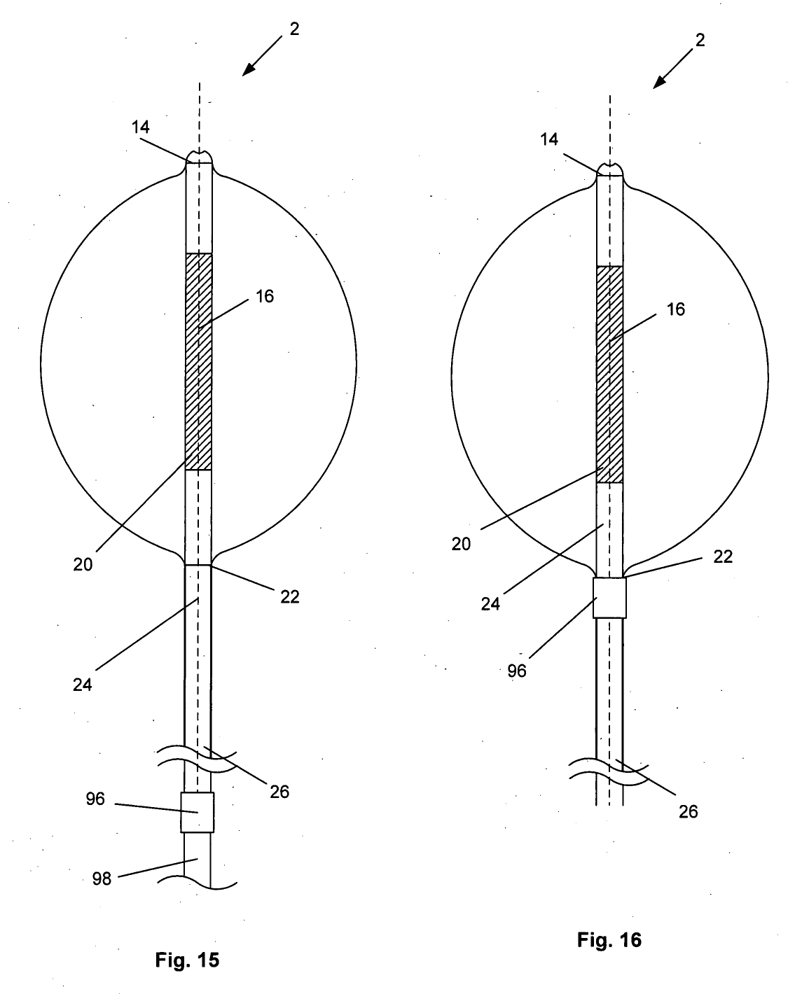

FIG. 15 illustrates that the catheter 26 can be removably or fixedly attached to a coupler 96. The coupler 96 can be removably or fixedly attached to a handle 98. The coupler 96 can be made from any material disclosed herein including rubber, elastic, or combinations thereof. The coupler 96 can have a substantially cylindrical configuration. The coupler 96 can have threads. The coupler 96 can have slots. The couple can have a joint and/or hinge 106.

The coupler 96 can be flexible. The coupler 96 can substantially bend, for example, permitting the longitudinal axis 16 of the handle 98 to be a substantially non-zero angle (e.g., from about 0° to about 90°) with respect to the longitudinal axis 16 of the catheter 26. The coupler 96 can permit substantially resistance free rotation between the longitudinal axis 16 of the catheter 26 and the longitudinal axis 16 of the handle 98.

FIG. 16 illustrates that the coupler 96 can be removably or fixedly attached to the catheter 26 on the proximal and distal end of the coupler 96. The coupler 96 can have and/or be proximally adjacent to the wire proximal sheath ports 22.

The measuring wire 100 can have a low and/or high friction surface. The measuring wire 100 can have a higher friction surface on the side of the measuring wire 100 radially exterior to the catheter 26 and a lower friction surface on the side of the measuring wire 100 radially interior to the catheter 26. The measuring wire 100 can have a surface having a substantially uniform friction around substantially the entire measuring wire 100.

The surface of the measuring wire 100 can be textured, for example knurled, pebbled, ridged, Toped, or combinations thereof. The surface of the measuring wire 100 can be textured on the side of the measuring wire 100 radially exterior to the catheter 26 and not substantially textured on the side of the measuring wire 100 radially interior to the catheter 26. The surface of the measuring wire 100 can be substantially uniformly textured around substantially the entire measuring wire 100.

The surface of the measuring wire 100 can be encrusted with a granulized material, for example diamond, sand, a polymer, the material from which the measuring wire 100 is made, any other material described herein, or combinations thereof. The surface of the measuring wire 100 can be encrusted on the side of the measuring wire 100 radially exterior to the catheter 26 and not substantially encrusted on the side of the measuring wire 100 radially interior to the catheter 26. The surface of the measuring wire 100 can be substantially uniformly encrusted around substantially the entire measuring wire 100.

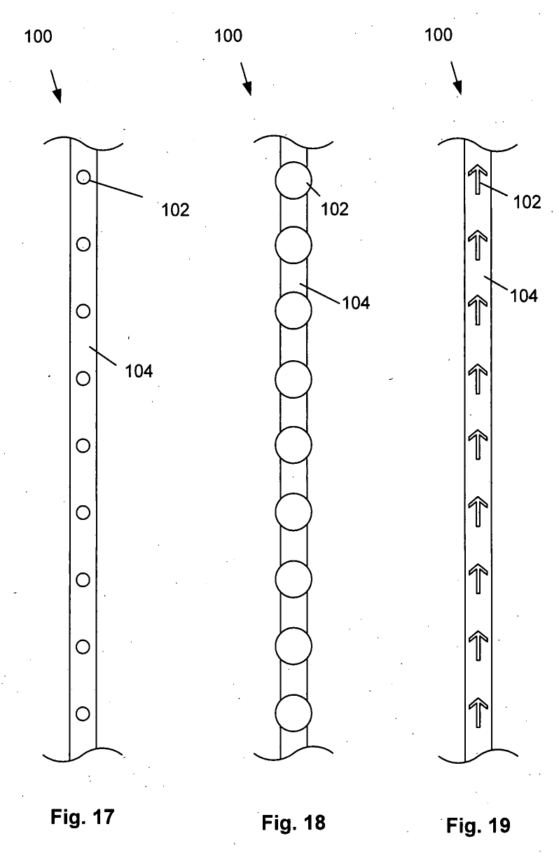

FIG. 17 illustrates that the measuring wire 100 can have a wire body 104 and one or more markers 102. The wire body 104 can have no markers 102. The markers 102 can be echogenic, radiopaque, magnetic, or configured to be otherwise visible by an imaging technique known to one having ordinary skill in the art. The markers 102 can be made from any material disclosed herein including platinum (e.g., pure or as powder mixed in glue).

The markers 102 can be uniformly and/or non-uniformly distributed along the length of the wire body 104. The markers 102 can be uniformly and/or non-uniformly distributed along the radius of the wire body 104. The markers 102 can be separate and discrete from the wire body 104. The markers 102 can be attached to the wire body 104. The markers 102 can be incorporated inside the wire body 104. The marker 102 can have configuration symmetrical about one, two, three, or more axes. The marker 102 can have an omnidirectional configuration. The marker 102 can have a configuration substantially spherical, ovoid, cubic, pyramidal, circular, oval, square, rectangular, triangular, or combinations thereof. The marker's 102 radius can be smaller than or substantially equal to the wire body's 104 radius at the location of the marker 102. FIG. 18 illustrates that the marker's 102 radius can be greater than the wire body's 104 radius at the location of the marker 102.

FIG. 19 illustrates that the marker 102 can have a unidirectional configuration. The marker 102 can be configured in the shape of an arrow. All or subsets of the markers 102 on a wire body 104 can be aligned, for example all of the unidirectionally configured markers 102 can be oriented in the same longitudinal or radial direction (e.g., distally, proximally) along the wire body 104.

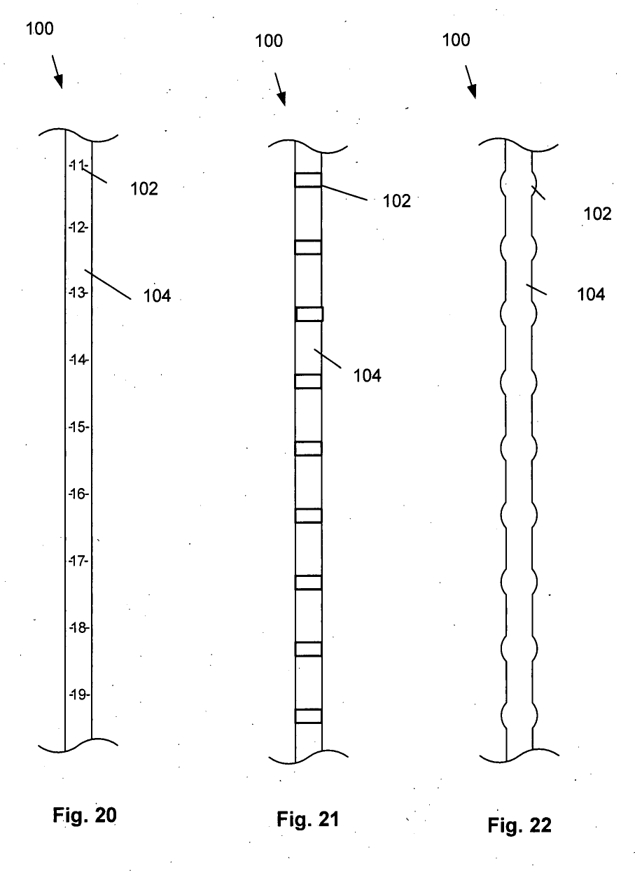

FIG. 20 illustrates that the markers 102 can have alphanumeric characters. The alphanumeric characters can increase in value (e.g., 1, 2, 3, or A, B, C, or I, II, III) incrementally along the length and/or radius of the wire. The markers 102 can include unit values (e.g., mm, in.)

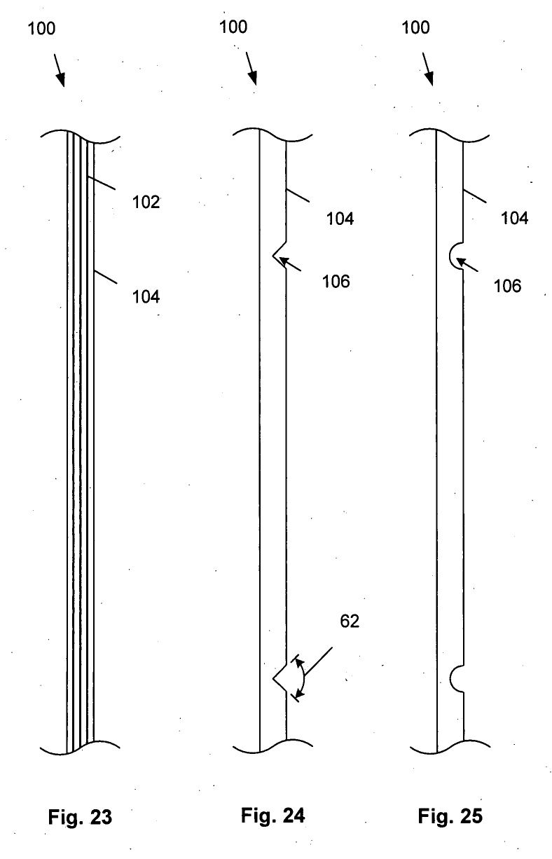

FIG. 21 illustrates that the markers 102 can be configured as a cylinder (e.g., disc), ring (e.g., toroid, band), partial cylinder, partial ring, or combinations thereof. FIG. 22 illustrates that the markers 102 can be integrated with the measuring wire 100. FIG. 23 illustrates that the markers 102 can be wires or threads. The markers 102 can extend along the length and/or radius of the wire body 104.

FIG. 24 illustrates that the wire body 104 can have one or more hinges 106. The hinges 106 can be configured to allow bending or other distortion of the wire body 104. The hinges 106 can be a change in material and/or a configuration. The hinge 106 can be configured by material absent from a side of the wire body 104. For example, the hinge 106 can be an angled cut (i.e., the angled cut is not necessarily cut. The angled cut can be cut, crimped, molded, etched, or combinations thereof) in the side of the wire body 104. The hinge 106 can have a stop to limit the bending of the measuring wire 100. For example, for an angle cut hinge 106, the stop can be the side of the hinge 106. The hinge 106 can have a hinge angle 62. The hinge angle 62 can correlate to the maximum angle of bending. The hinge angle 62 can be, as described elsewhere herein, or from about 1° to about 179°, more narrowly from about 15° to about 90°, yet more narrowly from about 20° to about 60°, for example about 45°.

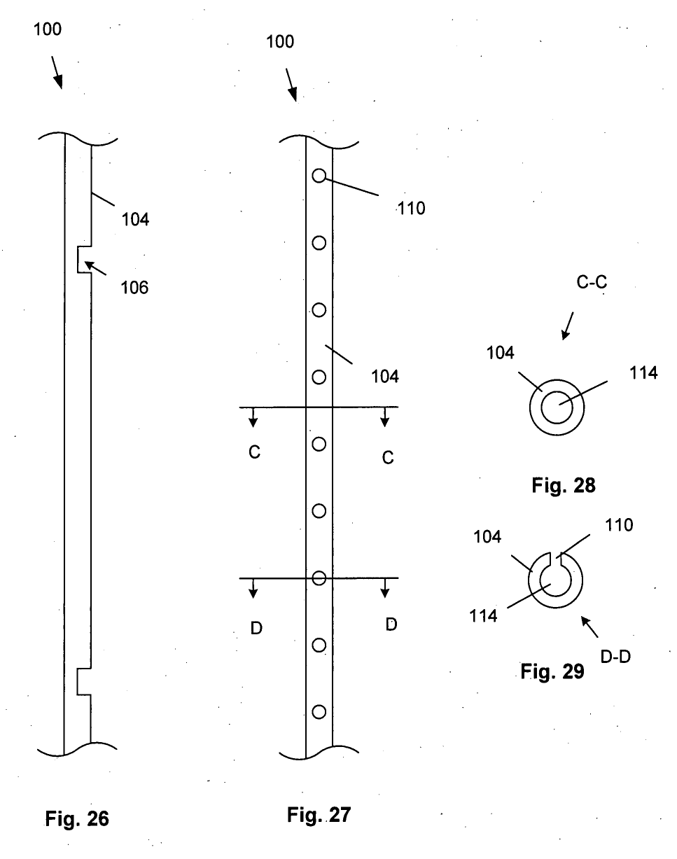

FIG. 25 illustrates that the hinge 106 can be a round cut. For example, the hinge 106 can be circular (e.g., semi-circular), oval, or combinations thereof. FIG. 26 illustrates that the hinge 106 can be a rectangular cut. For example, the hinge 106 can be rectangular (e.g., square). The hinge 106 can be any combination of the aforementioned configurations. The hinges 106 with various configurations can be on the same wire body 104. The hinges 106 can be on various sides of, or otherwise distributed at various angles around, the measuring wire 100.

FIGS. 27 through 29 illustrate that the wire body 104 can be hollow. The measuring wire 100 can have one or more wire conduits 114 on the radial interior of the wire body 104. The measuring wire 100 can have one or more wire conduit ports 110 in fluid communication with the one or more wire conduits 114 and the radial exterior of the measuring wire 100. The wire conduit ports 110 can regulate release of material inside of the wire conduits 114. For example, the wire conduit ports 110 (or wire conduits 114 themselves) can have and/or be filled and/or covered by an osmotic material, such as a matrix or film. The wire conduit ports 110 can all be on the same side of the measuring wire 100. The wire conduit ports 110 can be on various sides of, or otherwise distributed at various angles around, the measuring wire 100.

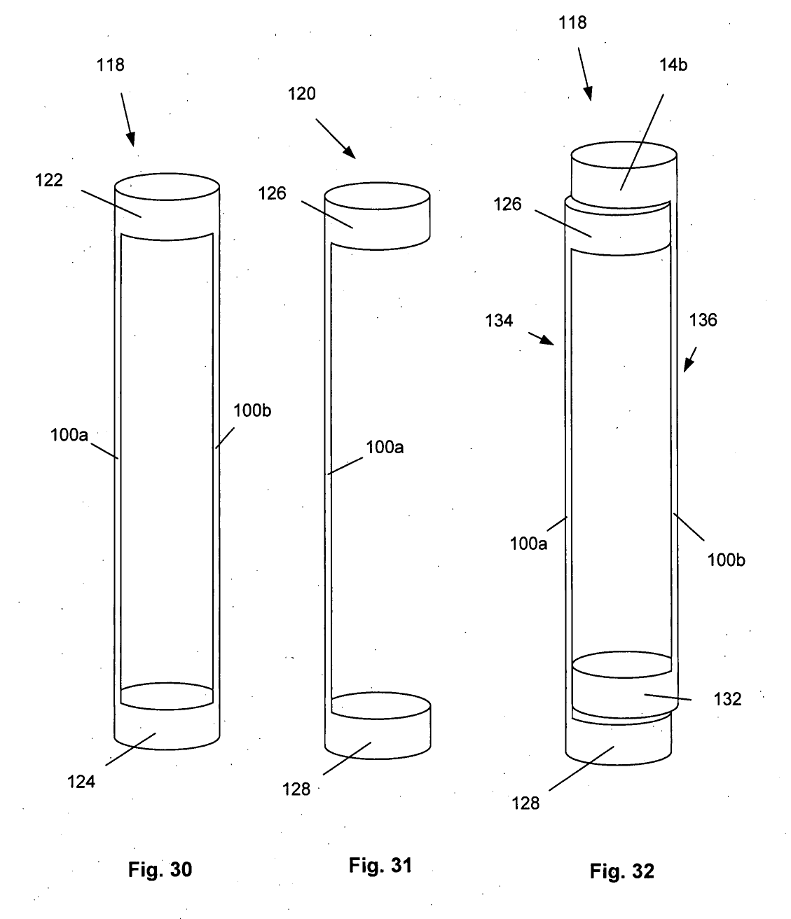

FIG. 30 illustrates that a wire assembly 118 can have a measuring wire 100 connected to one or more other elements. The first measuring wire 100a can be connected to the second measuring wire 100b at a distal collar 122 and/or a proximal collar 124. The first measuring wire 100a can be attached to and/or integral with the distal collar 122 and/or proximal collar 124. The second measuring wire 100b can be attached to and/or integral with the distal collar 122 and/or proximal collar 124. The wire assembly 118 can be made by being pressed, molded or cut from a tube, for example laser cut from a Nitinol tube. The collars can be cylindrical, have a rectangular, square, triangular, pentagonal, octagonal, oval cross section, or combinations thereof with respect to a longitudinal axis 16.

FIG. 31 illustrates that a wire sub-assembly 120 can have a first measuring wire 100a connected to one or more other elements. The first measuring wire 100a can be connected to a distal collar 122 and/or a proximal collar 124. The first measuring wire 100a can be attached to and/or integral with the distal collar 122 and/or proximal collar 124. The wire sub-assembly 120 can be made by being pressed, molded, or cut from a tube, for example laser cut from a Nitinol tube.

FIG. 32 illustrates that the wire assembly 118 can have a first wire sub-assembly 134 and a second wire sub-assembly 136. The first wire sub-assembly 134 and the second wire sub-assembly 136 can be integral and/or attached or separate. The first wire sub assembly can be positioned 180° opposite to the positioning of the second wire sub-assembly 136, with respect to a longitudinal axis 16 of the wire assembly 118.

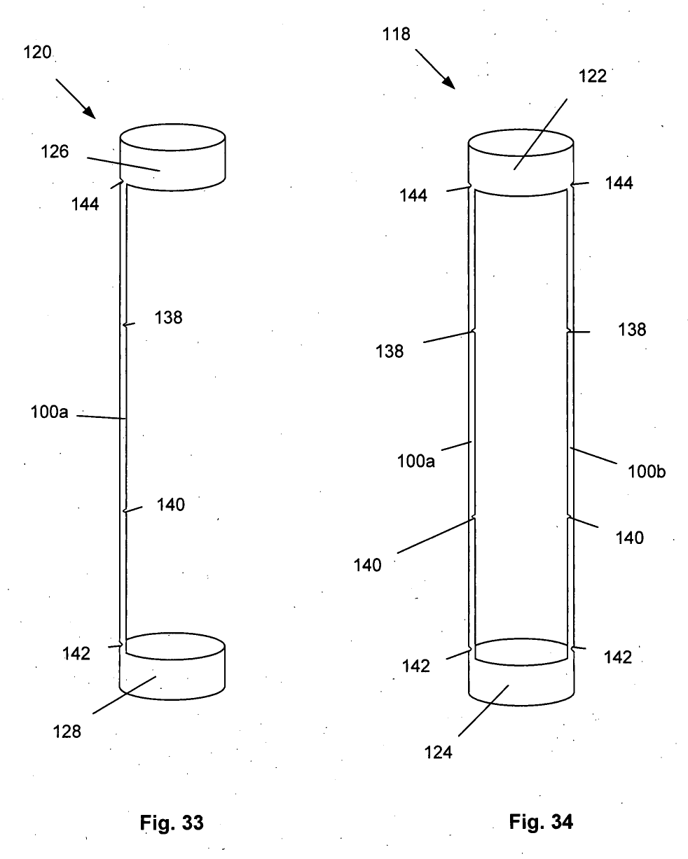

FIG. 33 illustrates that the wire assembly 118 can have a first measuring wire 100a that can have one or more hinges 106. For example, the first measuring wire 100a can have a wire distal hinge 144 and/or a wire proximal hinge 142. The wire distal hinge 144 can be at the connection between the first measuring wire 100a and the first wire distal collar 126. The wire proximal hinge 142 can be at the connection between the first measuring wire 100a and the first wire proximal collar 128. The wire distal hinge 144 and the wire proximal hinge 142 can be configured to bend or otherwise rotate the first measuring wire 100a radially outward from the central longitudinal axis 16 of the wire sub-assembly 120 when the measurement tool 2 is in a radially expanded configuration.

The wire can have a wire first hinge 138 and/or a wire second hinge 140. The wire first and/or second hinges can be on the first measuring wire 100a, for example, between the wire distal hinge 144 and the wire proximal hinge 142. The wire first hinge 138 and/or the wire second hinge 140 can be configured to bend or otherwise rotate the first measuring wire 100a radially inward from the central longitudinal axis 16 of the wire sub-assembly 120 when the measurement tool 2 is in a radially expanded configuration.

FIG. 34 illustrates that the wire assembly 118 can have wire distal hinges 144, and/or wire proximal hinges 142, and/or wire first hinges 138, and/or wire second hinges 140 on the first measuring wire 100a and/or the second measuring wire 100b.

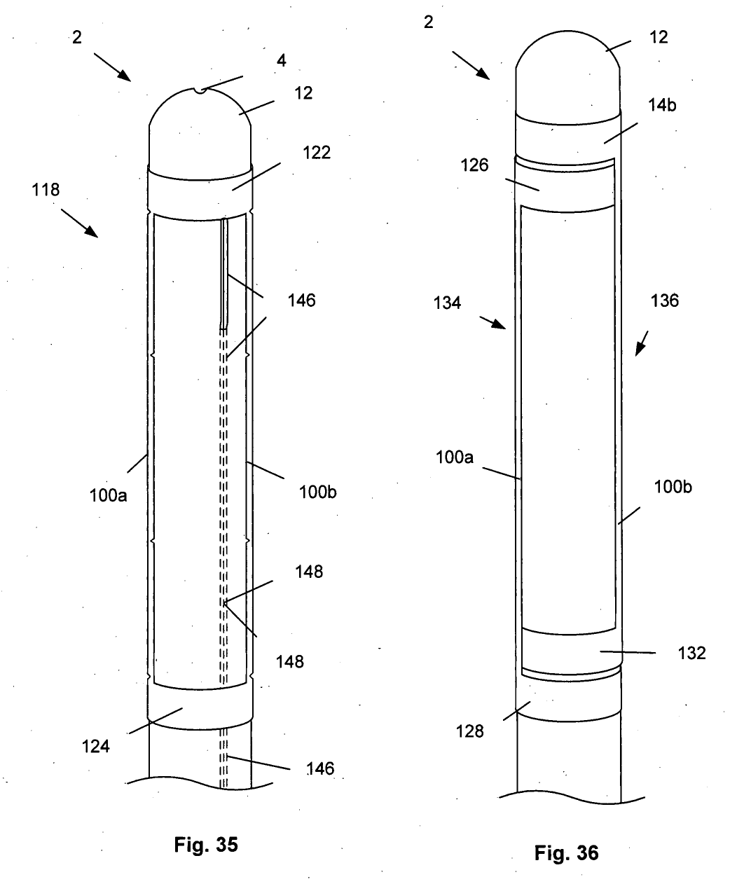

FIG. 35 illustrates that the measurement tool 2 can have a wire assembly 118 connected to the catheter 26. The wire assembly 118 can be integrated and/or attached to the catheter 26. For example, the proximal collar 124 and/or distal collar 122 can be integral with and/or fixably and/or slidably attached to the catheter 26. For example, the distal collar 122 can be slidably attached to the catheter 26 near or on the tip 12 and/or the proximal collar 124 can be fixedly attached to the catheter 26.

The wire assembly 118 can have a retraction leader 148. The retraction leader 148 can be integral with or attached to the distal collar 122. The retraction leader 148 can be rigid and/or flexible. The retraction leader 148 can be radially external to the catheter 26 and/or the retraction leader 148 can be slidably attached to a retraction leader conduit 146 or channel inside of the catheter 26. The retraction leader conduit 146 or channel can be partially or completely open to the radial outside of the catheter 26. For example, the retraction leader conduit 146 can be open to the radial outside of the catheter 26 for all or part of the retraction leader conduit's 146 length distal to the proximal conduit.

FIG. 36 illustrates that the first wire sub-assembly 134 and the second wire sub-assembly 136 can be integral with and/or attached to the catheter 26. The first wire sub-assembly 134 can be proximal, distal, or overlapping with the longitudinal position of the second wire sub-assembly 136 on the catheter 26. The second wire distal collar 130 can be distal and/or proximal to the first wire distal collar 126. The second wire proximal collar 132 and be distal and/or proximal to the first wire proximal collar 128. The first wire distal collar 126 can be attached to or integral with a first retraction leader 148. The second wire distal collar 130 can be attached to or integral with a second retraction leader 148.

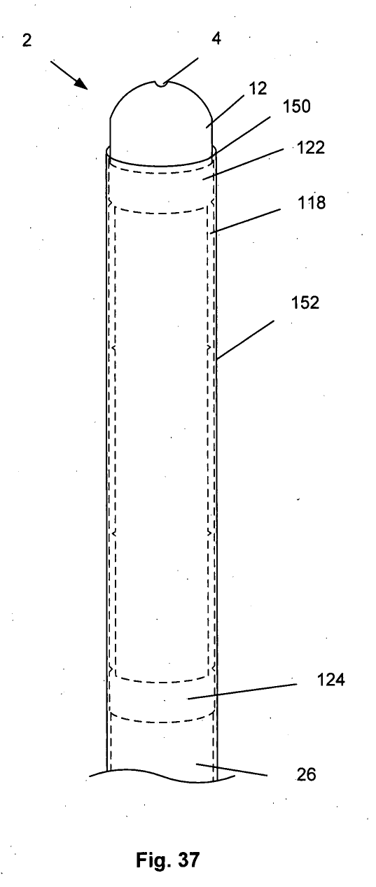

FIG. 37 illustrates that the measurement tool 2 can have a catheter sheath 152. The catheter sheath 152 can be slidably attached to the catheter 26. In an undeployed configuration, the catheter sheath 152 can be radially outside and longitudinally overlapping the wire assembly 118. The catheter sheath 152 can be sufficiently rigid to retain the wire assembly 118 in a radially contracted configuration. The catheter sheath 152 can have, for example at a distal end of the catheter sheath 152, a catheter sheath port 150 through which the catheter 26 and other elements (e.g., the wire assembly and measuring wires), can exit and enter the catheter sheath 152.

Any or all elements of the measurement tool 2 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, Del.), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue 154, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the measurement tool 2 and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The measurement tool 2 and/or elements of the measurement tool 2 and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

### Methods of Use

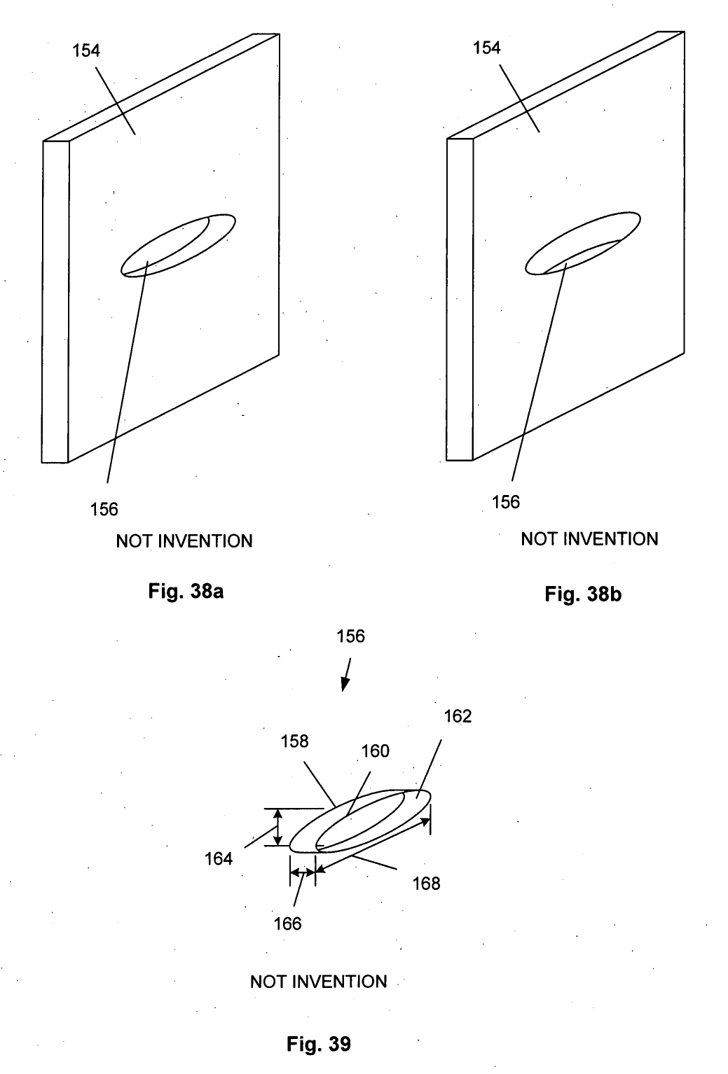

FIG. 38a illustrates a section of tissue 154 that can have a tunnel defect 156 passing through the tissue 154. The tunnel defect 156 can be substantially perpendicular to the face of the tissue 154. For example, the tunnel defect 156 can be an atrial septal defect (ASD). FIG. 38b illustrates that the tunnel defect 156 can be at a steep angle or substantially parallel to the face of the tissue 154. For example, the tunnel defect 156 can be a patent foramen ovale (PFO).

FIG. 39 illustrates that the tunnel defect 156 can have a defect front face 162 and a defect back face (not shown). A defect front lip 160 can be defined by the perimeter of the defect front face 162. A defect back lip 158 can be defined by the perimeter of the defect back face. The tunnel defect 156 can have a defect height 164, a defect depth 166 and a defect width 168.

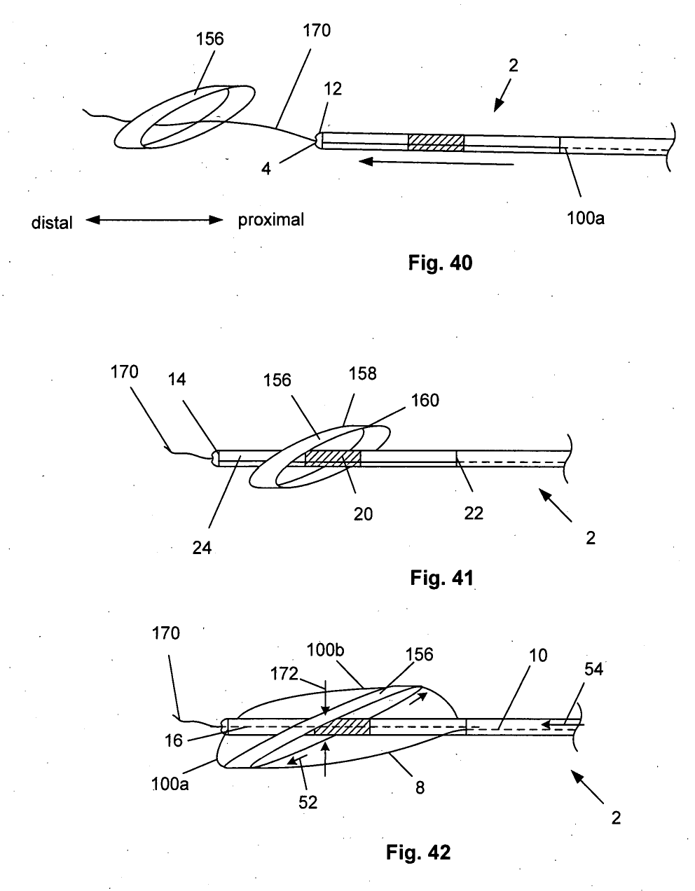

FIG. 40 illustrates that a guidewire 170 can be deployed through the tunnel defect 156. The guidewire 170 can be passed through the guide lumen 4 in the measurement tool 2. The measurement tool 2 can be in a radially contracted (as shown) or radially expanded configuration. The measurement tool 2 can be translated, as shown by arrow, along the guidewire 170. The measurement tool 2 can be translated to the tunnel defect 156 with or without the use of the guidewire 170.

FIG. 41 illustrates that the measurement tool 2 can be translated into the tunnel defect 156. The guidewire 170 can be left in place or removed. The location of the measurement tool 2 can be monitored by dead reckoning, and/or imaging, and/or tracking along the length of the guidewire 170. The measurement tool 2 can be positioned so that the tunnel defect 156 is located adjacent to the catheter porous section 20. The measurement tool 2 can be positioned so that the tunnel defect 156 is located substantially between the most distal wire distal anchor 14 and the most proximal wire proximal sheath.

FIG. 42 illustrates that the measurement tool 2 can be radially expanded. The measuring wires 100 in the wire radially constrained section 10 can be distally longitudinally translated, as shown by arrow 54. The measuring wires 100 can translate radially (i.e., away from the longitudinal axis 16), as shown by arrows 52. The measuring wires 100 can radially distend the tunnel defect 156, for example causing the tunnel defect 156 to widen (shown by arrows similar to arrows 52) and shorten or otherwise contract in height, as shown by arrows 172. The measuring wires 100 can radially distend the tunnel defect 156, for example, until the tunnel defect 156 will no longer distend without structurally damaging the tunnel defect 156.

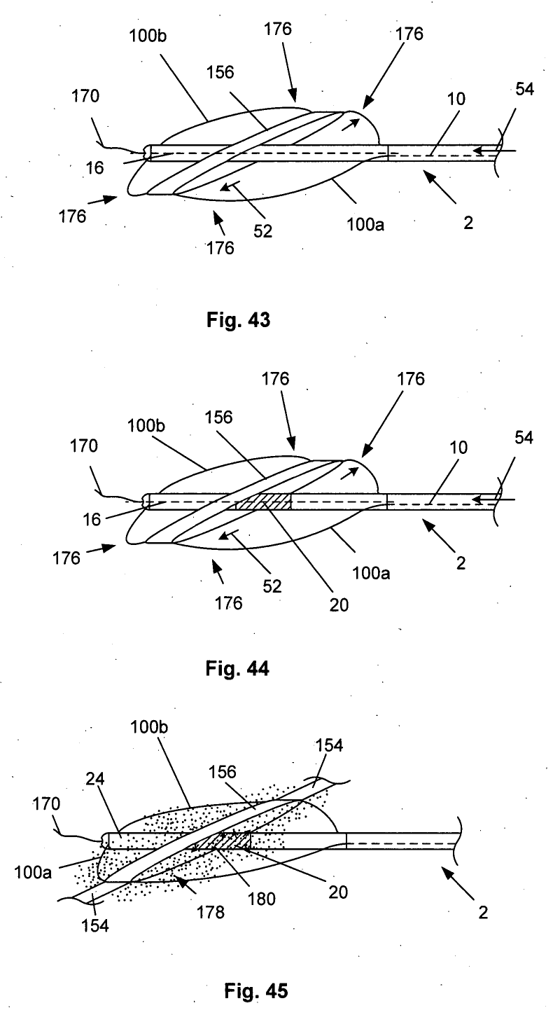

FIG. 43 illustrates that the measuring wires 100 can be radially translated beyond the extent that the tunnel defect 156 can be distended without structural damage. The measuring wires 100 can deform around the front and back defect lips. Portions of the measuring wires 100 can configure into wire overdeployment sections 176 proximal and distal to the tunnel defect 156. The wire overdeployment sections 176, or markers 102 thereon, can be imaged, for example using x-rays (e.g., radiography, fluoroscopy), ultrasound, or magnetic resonance imaging (MRI). The wire overdeployment sections 176 can illustrate the defect width 168 (i.e., the length between the wire deployment sections) when the defect is in a fully distended configuration.

FIG. 44 illustrates that the measurement tool 2 can have no catheter porous section 20, for example, when the measurement tool 2 is used for the measurement method as shown in FIG. 43. The methods of use shown in FIGS. 43 and 44 can, for example, measure the defect depth 166 and/or the defect height 164.

FIG. 45 illustrates that contrast fluid or particles can be deployed into the fluid lumen 36 of the catheter 26, for example, when tunnel defect 156 is in a fully distended configuration. The contrast fluid can be radiopaque, echogenic, visible contrast (e.g., dyes, inks), any other material disclosed herein, or combinations thereof. The fluid pressure of the contrast fluid or particles can be increased. The contrast fluid or particles can emit, as shown by arrows 180, through the catheter porous section 20. The contrast fluid or particles outside of the catheter 26 can configure into a marker cloud 178. The marker cloud 178 can move into position around the tissue 154. The marker cloud 178 can illustrate the defect dimensions (i.e., visible with imaging systems known to those having ordinary skill in the art, including x-ray, CAT, MRI, fiber optic camera, ultrasound/sonogram) when the defect is in a fully distended configuration.

A drug can be deployed from the catheter porous section 20, for example, similar to the method of deploying the contrast fluid.

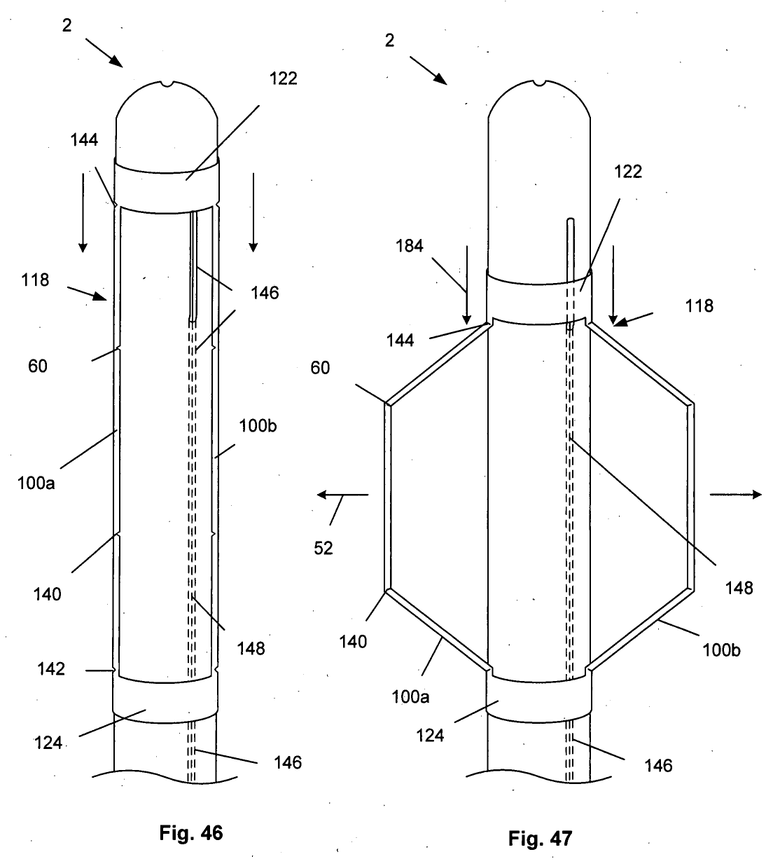

FIG. 46 illustrates that a proximal force, as shown by arrows, can be applied to the distal collar 122. For example, the retraction leader 148 can be pulled proximally.

FIG. 47 illustrates that the distal collar 122 can translate proximally, as shown by arrows 52. The measuring wires 100 can expand radially away from the central longitudinal axis 16 of the measurement tool 2. The wires can bend radially outward at the wire distal hinge 144 and the wire proximal hinges 142. The wires can bend radially inward at the wire first hinge 138 and wire second hinge 140. The wires can also form a curved or splined configuration (e.g., similar to the configuration shown in FIG. 6, inter alia) instead of or in addition to the hinges 106.

The measuring wires 100 can be resiliently biased to the radially contracted configuration. When the proximal force is no longer applied to the distal collar 122, the measuring wires 100 can straighten and distally force the distal collar 122 to translate to the position shown in FIG. 46.

The measurement wires can be deformable. The retraction leader 148 can be rigid. For example, to radially contract the measuring wires 100, the retraction leader 148 can distally force the distal collar 122 to translate to the position shown in FIG. 46. The measuring wires 100 can deform to the position shown in FIG. 46.

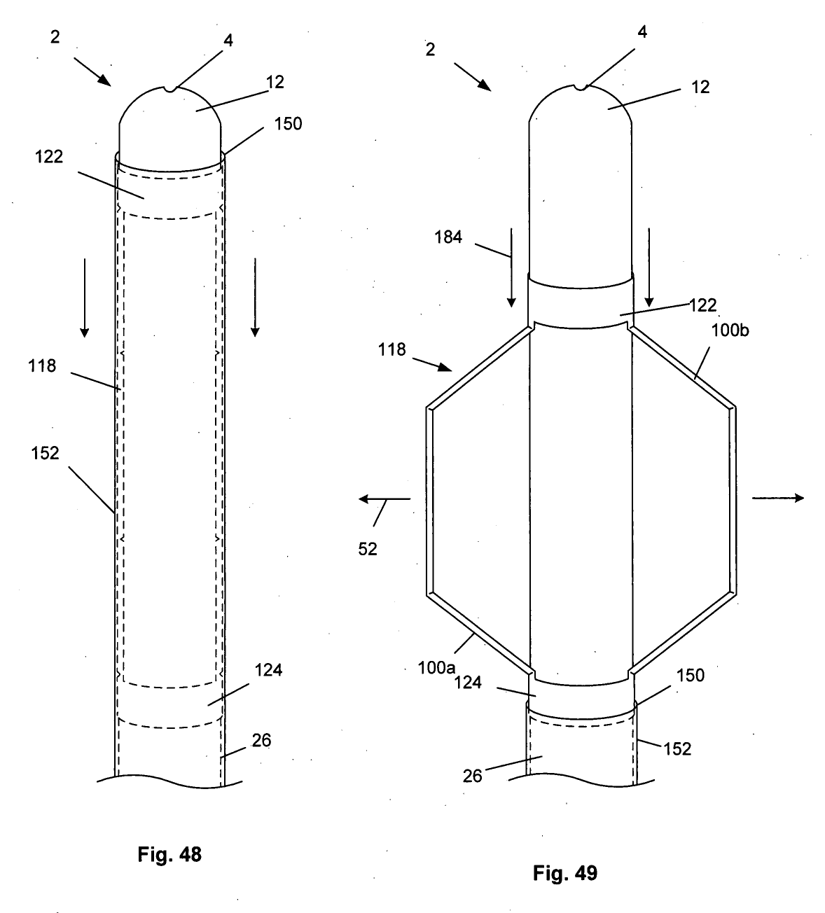

FIG. 48 illustrates that the wire assembly 118 can be radially constrained by the catheter sheath 152. The catheter sheath 152 can radially encircle the measuring wires 100 and/or the entire wire assembly 118. The catheter sheath 152 can longitudinally encompass the measuring wires 100 and/or the entire wire assembly 118. A distal force, as shown by arrows, can be applied to the catheter sheath 152.

FIG. 49 illustrates that the measuring wires 100 can be resiliently biased to radially expand away from the center longitudinal axis 16 of the measurement tool 2. When the catheter sheath 152 is retracted distal to the measuring wires 100, the measuring wires 100 can radially expand, as shown by arrows. The distal collar 122 can proximally translate, as shown by arrows.

The catheter sheath 152 can be rigid. The catheter sheath 152 can be distally translated, for example to radially contract the measuring wires 100. The catheter sheath 152 can radially contract the measuring wires 100 as the catheter sheath 152 substantially underformably slides distally over the measuring wires 100.

A distension device size can be determined as described, supra. The measurement tool 2 can be radially contracted and removed from the tunnel defect 156, or the coupler 96 and/or the elements of the measurement tool 2 proximal to the coupler 96 can be detached from the remainder of the measurement tool 2 and removed. If the entire measurement tool 2 is removed from the tunnel defect 156, a distension device can be selected that has a size that substantially matches (e.g., is equivalent when the distension device is in a substantially or completely radially expanded configuration) the size of the distended tunnel defect 156. The distension device can be deployed to the tunnel defect 156, for example along the guidewire 170. The guidewire 170 can be removed. The distension device can be, for example, a filter, stopper, plug, any distending device described in U.S. patent application Ser. No. 10/847,909, filed 19 May 2004; Ser. No. 11/184,069, filed 19 Jul. 2005; and Ser. No. 11/323,640, filed 3 Jan. 2006, all of which are incorporated by reference herein in their entireties, or any combinations thereof.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

We claim:

1. A device having a longitudinal axis, wherein the device is for measuring the width of a distended defect in tissue, the device comprising:
a first elongated member, and wherein the first elongated member is configured to expand away from the longitudinal axis.

2. The device of claim 1, further comprising a second elongated member, wherein the first elongated member is opposite with respect to the longitudinal axis to the second elongated member, and wherein the second elongated member is configured to expand away from the longitudinal axis.

3. The device of claim 2, further comprising a lumen.

4. The device of claim 3, further comprising a porous cover on the lumen.

5. The device of claim 2, further comprising a catheter, wherein the lumen is in the catheter.

6. The device of claim 1, wherein the catheter is fixedly attached to the first elongated member.

7. The device of claim 1, wherein the catheter is slidably attached to the first elongated member.

8. The device of claim 1, wherein the first elongated member is substantially flexible.

9. The device of claim 1, wherein the second elongated member is substantially flexible.

10. The device of claim 1, further comprising a coupler, wherein the coupler is attached to the catheter.

11. The device of claim 1, wherein the first elongated member comprises a wire body and a marker.

12. A method for sizing a tunnel defect, comprising:
inserting a measurement tool into the tunnel defect,
distending the tunnel defect into a distended configuration, and
measuring the tunnel defect in the distended configuration.

13. The method for claim 12, wherein distending comprises radially expanding the measurement tool.

14. The method for claim 12, wherein measuring comprises bending the first measuring wire around a front lip of the tunnel defect.

15. The method for claim 14, wherein measuring comprises bending the first measuring wire around a rear lip of the tunnel defect.

16. The method for claim 12, wherein measuring comprises emitting a contrast fluid in the tunnel defect.
